(19) United States
(12) Patent Application Publication
(10) Pub. No.: US 2022/0222622 A1
(43) Pub. Date: Jul. 14, 2022

(54) HEALTHCARE INVENTORY MANAGEMENT DISTRIBUTED LEDGER SYSTEM AND METHOD

(71) Applicant: TYDEi Health, Inc., Glastonbury, CT (US)

(72) Inventors: Charles A. Weidman, IV, Glastonbury, CT (US); John Hatchell, Somers, CT (US)

(73) Assignee: TYDEi Health, Inc., Glastonbury, CT (US)

(21) Appl. No.: 17/480,097

(22) Filed: Sep. 20, 2021

Related U.S. Application Data
(60) Provisional application No. 63/136,625, filed on Jan. 12, 2021.

(51) Int. Cl. G06Q 10/08; G16H 40/20; G06Q 20/38

(52) U.S. Cl. CPC G06Q 10/0875; G06Q 2220/00; G06Q 20/38215; G16H 40/20

(57) ABSTRACT

A healthcare inventory management distributed ledger is a computer-implemented system and method for a cryptographic ledger application (CLA) that improves the inventory management, and financial transactions and process of communication between a healthcare facility and any vendor or third-party supplier organization that supplies for products for use within a facility. The healthcare CLA disclosed herein allows the facility, vendor, or third-party payors to transact on a common ledger system for an official record, communication, management, reconciliation, and remittance. Moreover, the healthcare CLA permits parties to use this system for product that is not the property of the facility.

### CROSS-REFERENCE TO RELATED APPLICATION

This application claims the benefit of U.S. Provisional Application Ser. No. 63/136,625, filed Jan. 12, 2021, which is incorporated herein by reference in its entirety.

### TECHNICAL FIELD

The present invention generally relates to the healthcare field, and more particularly, to the methods and systems for data management using a distributed ledger.

### BACKGROUND

Healthcare facilities frequently require an inventory of products from third parties and vendors to perform necessary surgical procedures. The inventory usually comes at a significant expense, and traditional models for inventory management are difficult to utilize due to cost and efficiency. Currently, most healthcare entities utilize supply on an “as needed” basis, meaning that when a surgical procedure is scheduled, a healthcare facility will make a formal request, or transaction, to a vendor or third-party to procure product(s) to use for the procedure. Some of the third-party and vendor organizations who transact with healthcare facilities in this manner will leave inventory supply at the facilities, to avoid incurring distribution costs. This is known as “consignment inventory” and is a common practice and conventional in the field.

At completion of a surgical procedure, use of a product is logged in the health care entity or provider's records, typically electronically, but only on behalf of the entity or provider. The official record of these transactions on behalf of the vendor or third-party organization is then conventionally transcribed on paper, with specific patient, provider, and facility information. Most vendors or third-party organizations use identification stickers, included in the implant packaging, to properly identify the product when recording the use. The stickers of implants or other products that were used in a procedure are attached to the paper record. If there are no identification stickers, someone present at the procedure conventionally records all items used manually, using identification numbers and descriptions. Once all items have been logged, the record is then authorized by signature via a staff member. This verification attempts to ensure the accuracy of products used by someone who serves as a witness in the surgical room. When completed, these records are returned to the vendor or third-party organization in whatever manner the healthcare facility chooses.

A purchase order (PO) is eventually received by the vendor or third-party for products used by provider and facility, however, often a vendor is expected to restock products, especially implants, even before a PO is executed. The vendor or third-party will then issue a conventional paper invoice for payment to the appropriate party within the facility, who will forward it to one or more payees, such as insurance companies or patients. This paper record serves as the common source of reference for remittance and reconciliation.

These prior paper-based systems are costly, inefficient, prone to errors, subject to incomplete, inaccurate or lost records, miscommunication, and delays. Further, it common to have monthly accounts receivables of 10-15 percent in the industry because of the paper-based purchase order process and the need to receive those paper documents before a sale is recorded.

The current systems negatively impact vendor organizations through high customer service costs, constant communication to hospital accounts (which drains resources and time), and challenges in reconciliation. It is not conventional in the art for a healthcare facility, vendor, and/or thirty-party to us a common ledger system for an official record, communication, management, reconciliation, and remittance and thus, problems with prior inventory management systems and payment management systems are common. Current systems also do not protect patient information.

### SUMMARY

In a first aspect, a computer-implemented method for managing product inventory using a distributed ledger includes receiving inventory data comprising a product list from a vendor; storing the inventory data as records in the distributed ledger; executing a first smart contract in the distributed ledger to process a request to use a selected product from the product list; executing a second smart contract in the distributed ledger to record the completion of use of the selected product; and executing a third smart contract to complete a payment process to the vendor.

In a second aspect, a system for healthcare inventory management for managing product inventory using a distributed ledger, the system comprising a processor and a data store storing data and instructions which, when executed, cause the processor to receive inventory data comprising a product list from a vendor; store the inventory data as records in the distributed ledger; execute a first smart contract in the distributed ledger to process a request to use a selected product from the product list; execute a second smart contract in the distributed ledger to record the completion of use of the selected product; and execute a third smart contract to complete a payment process to the vendor.

In a third aspect, the method or system may provided on a non-transitory computer-readable medium for execution by a processor.

In any of the above aspects, executing the first smart contract includes checking the inventory data records in the distributed ledger to see if the selected product is available for use.

In any of the above aspects, executing the second smart contract includes scanning a barcode on the selected product before it is used in a surgical procedure. Further, executing the second smart contract includes verifying that the selected product is appropriate for the surgical procedure. Yet further, executing the second smart contract includes scanning the barcode on the selected product after the surgical procedure; and storing a record in the distributed ledger.

In any of the above aspects, executing the third smart contract includes generating a purchase order; creating an invoice based on the purchase order; and updating the inventory data records in the distributed ledger.

In any of the above aspects, encrypting all records before they are stored in the distributed ledger.

### BRIEF DESCRIPTION OF THE DRAWINGS

The accompanying drawings, which are incorporated in and constitute a part of the specification, illustrate exemplary embodiments of the invention. Together with the description that follows, the drawings serve to explain the Objects, advantages, and principles of the invention. In the drawings:

FIG. 1 is a block diagram of a healthcare inventory management distributed ledger system, in embodiments.

FIG. 2 is a block diagram of a representative process flow for a healthcare inventory management distributed ledger system, in embodiments.

FIGS. 3A-3C illustrate methods for use with a healthcare inventory management distributed ledger system, in embodiments.

FIG. 4 illustrates a representative product safety process as part of a healthcare inventory management distributed ledger system, in embodiments.

FIG. 5 illustrates a representative purchase order as part of a healthcare inventory management distributed ledger system, in embodiments.

FIG. 6 illustrates a representative recall process as part of a healthcare inventory management distributed ledger system, in embodiments.

### DETAILED DESCRIPTION OF PREFERRED EMBODIMENTS

Reference will now be made in detail to exemplary embodiments of the invention, some aspects of which are illustrated in the accompanying drawings.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the claims. Furthermore, there is no intention to be bound by any expressed or implied theory presented in the preceding technical field, background, summary, or the following detailed description.

A healthcare inventory management distributed ledger, in an embodiment, is a computer-implemented system and method for a cryptographic ledger application (CLA) that improves the inventory management, and financial transactions and process of communication between a healthcare facility and any vendor or third-party supplier organization that supplies product for use within a facility. The healthcare CLA disclosed herein allows the facility, vendor, or third-party to transact on a common ledger system for an official record, communication, management, reconciliation, and remittance. Moreover, the healthcare CLA permits parties to use this system for product that is not the property of the facility. The terms “distributed ledger” and “cryptographic ledger application” may be used interchangeably herein.

In another embodiment, the disclosed system allows users to create specific accounts for each party involved in a transaction. A party may include any facility, vendor, third-party, other entity, and designated persons with access at those organizations who might be coordinating business among the various parties. The data may be individually input into the application, may be related to specific individual account, and may allow for an automated information flow specific to a business transaction via smart contract language in the system.

In a further embodiment, each “event” may be specifically notated by a unique identifier or other information. For example, an event may be identified by names, dates, numbers, site specific information, or other identifiers so that a digital record can be easily located and understood. Some information, specifically as it pertains to patients being served in a healthcare facility, may be sourced directly through an Electronic Medical Record (EMR), Electronic Health Record (EHR), or Fast Healthcare Interoperability Resource (FHIR) server system. During an “event” where a facility may utilize product inventory, a representative of the facility may log product usage via a scanning feature or manually log products on the digital record specific to the event. Due to the immutability of the solution, it is preferable to verify information by designated representative prior to submission.

Once submitted, smart contract language will then automatically process the digital record in accordance with facility specifications. Private information such as Personally Identifiable Information (PII) and Protected Health Information (PHI) may be cryptographically encrypted to ensure security in the system. Smart contracts will take effect once certain conditions or contract parameters between a facility and other organization have been met. Conditional requirements may be linked specifically to product inventory that is either “owned,” “consigned,” or “loaned” on an as needed basis by the facility. In an embodiment where product inventory is considered “on consignment” or “loaned,” the healthcare CLA can provide visibility on quantity, description, expiration, price, etc. between the facility and “vendor” or “third party.” The system may also alert a facility representative if a product being used is expired or not compliant during an “event” as well as pre-authorize a product prior to utilization in an “event.”

In one embodiment, if the product submission form related to the “event” meets certain smart contract conditions, a purchase order will automatically be processed. In another embodiment when certain smart contract conditions are met, an automated invoice may be processed through an Enterprise Resource Planning (ERP) system that is integrated with the facility. The products used by the facility can also be automatically re-ordered and delivered to their location or designation. In embodiments, smart contracts may also include FDA device indications. If an item is entered into the system that does not follow the FDA's requirements for a procedure, the system will notify the surgical room in order to prevent an item from being used that does not belong to that patient.

In another aspect, communication regarding any individual transaction between the parties can be facilitated through a secure messenger feature in the application. Messages may be captured and stored in the CLA, creating a communication portal that eliminates conversation in non-trusted formats.

Data related to transaction records between a facility, a vendor, or a third-party supplier may be accessible through the account's application portal. Each party may have full visibility of all accumulated records and data points.

Further aspects may allow hospitals and vendors to permit payers to access and transparently view information in the secure management system. The system may include an application that connects hospitals, vendors, and payers in one transparent and secure ecosystem. The interconnectivity of all the entities involved in the purchase, storage, distribution, and use of implants reduces cost and time in the inventory management process while providing a system of shared accountability. The disclosed invention improves safety, improves security of controlling PHI/PII, creates transparent visibility by parties with access, permits immutable records for use, audit, and reconciliation of real-time data related to product use.

A healthcare distributed ledger system used by healthcare facilities, vendors and other participants in the healthcare system promotes efficient purchase orders, trustworthy payments, and prompt payments. In another embodiment, the system may promote transparent product pricing that is visible by any party, allow for auditable reimbursement of claims related to products used, and permit the ability to correlate product usage data to procedures (via simple coding as compared with complex healthcare coding references). This disclosed ledger system saves payers significant money and improves the verification and accuracy of data within the system.

The disclosed application allows communication, including communication pertaining PHI/PII, to be secured and protect by the cryptography of the healthcare CLA. The system will ensure the security of patient health information through immutable records of all communications and transactions. In one embodiment, cryptographic encryption of PHI/PII may be employed to prevent vendor mishandling of the healthcare information. Smart contracts on a ledger-based application provide for these transactions to be automated based on conditional requirements that are contracted between all parties. In another aspect, the application can individually account for any endorsements. The immutable records of utilization can also be used as a registry for each healthcare entity who adopts the application. As a registry, the benefit of locating specific patients with implanted products that are part of a recall can easily be achieved, for example through a dashboard utility.

A further aspect of the invention may promote a more cost-efficient implant or product usage for the healthcare system through increased payor involvement and transparency in product use decisions. A distributed ledger shared between all parties may allow for the payor to generate payment for implant costs associated with a claim directly to the vendor as opposed to going through the hospital or facility that purchases the implants from the vendor. This system would save time, money and human capital. Payor arrangements are currently structured with premiums built into hospital implant reimbursement. Removing those costs would reduce the overall cost burden for payor, reduce cost passed down to consumers and free more money for claims too often denied; a benefit to all participants in the US healthcare system.

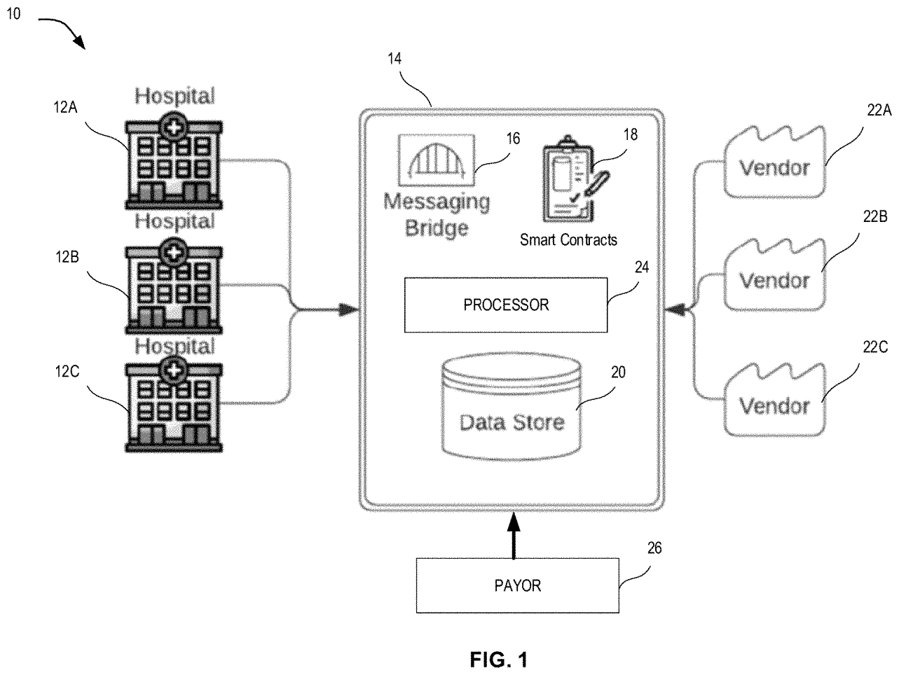

FIG. 1 is a block diagram of a healthcare inventory management using a distributed ledger system 10, in embodiments. Healthcare facilities 12A, 12B and 12C interact with vendors 22A, 22B and 22C and payor 26 using distributed ledger 14. Healthcare facilities 12A, 12B and 12C are depicted as hospitals, but may represent any type of facility where patients receive care, including outpatient centers and rehab facilities. Although three facilities are pictured, any number of healthcare facilities may use system 10. Similarly, vendors 22A, 22B and 22C are not limited to three and may represent any entity that creates or sells products that may be used in a healthcare environment. This may include products used directly with a patient such as an implant, or other tools or products used by hospital personal, such as tools or consumables. Distributed ledger 14 includes a processor 24 for executing instructions to provide the functionality described herein. Data store 20 stores instructions for execution by processor 24. Data store 20 also stores data of transactions executed in system 10 and smart contracts 18, which may also be executed by processor 24. Distributed ledger 14 may also include messaging bridge 16, which will be discussed further below.

Information requests, data updates and system calculations are processed by system 10 based on the rights and obligations described by the owners of smart contracts 18. Additionally, the processing of a smart contract is described by the logic and rules embedded in its code.

In embodiments, smart contracts 18 will perform a variety of functions in system 10. For example, a create function creates a new contract with a set of data parameters and sets the status of the new contract to “Active.” A fetch action checks the existence of and retrieves data from a smart contract. An exercise action executes an action on smart contract 18 resulting in a transaction of data using the smart contract. Other types of functions and smart contracts are contemplated as needed to provide the embodiments disclosed herein. Additionally, system 10 may store the outcome of execution of smart contracts 18 in a variety of distributed ledger platforms, centralized ledgers or even databases.

In embodiments, processor 24 represents any computer-implemented device or method. It may represent multiple computer-implemented devices. It may be physically located with data store 20, or provided in a cloud-computing or similar type of system. Messaging bridge 16 may be executed by processor 24 or provided as a separate service that interacts with distributed ledger 14 and processor 24. Payor 26 represents insurance companies or other institutions responsible for the payment process.

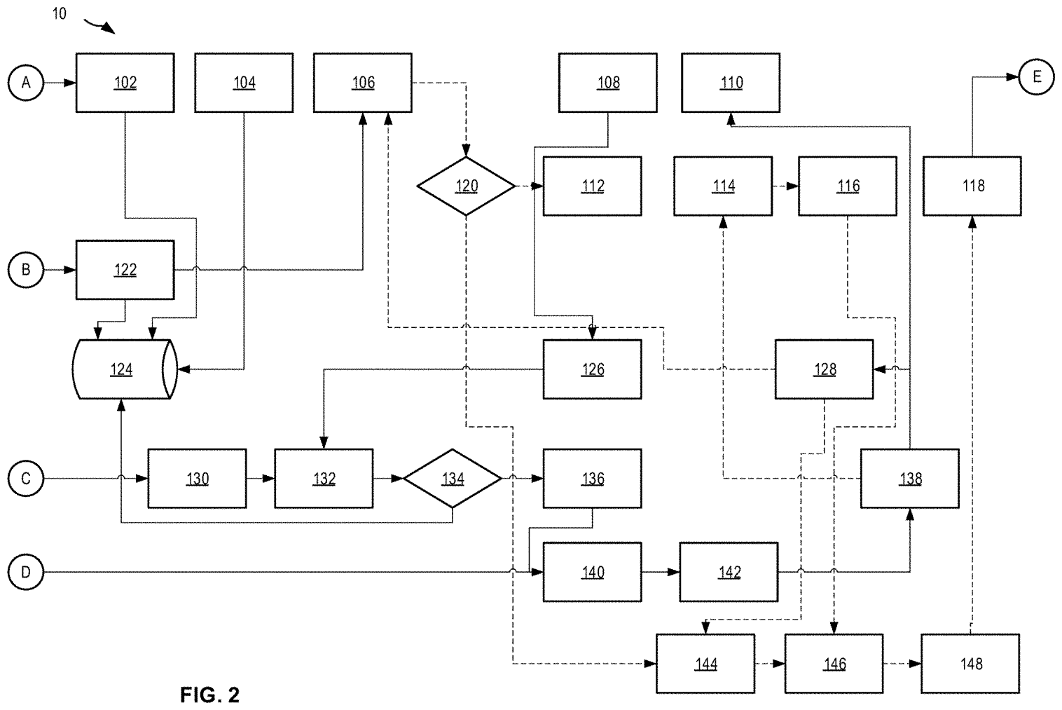

FIG. 2 is a block diagram of a representative process flow 100 for a healthcare inventory management system 10, in embodiments. Process flow 100 is shown to illustrate principles disclosed herein, but variations may be made from the specific processes discussed below. In embodiments, solid lines represent a business flow while dashed lines represent a financial process flow.

System 10 will connect to any payor 26 to create an ecosystem between hospital, surgical or general healthcare facility and vendor suppliers. Through access payor 26 will have transparency to specific data sets that are relevant to an insurance claim. These data sets include, but are not limited to: all implant or product information relevant to a particular claim, procedures, etc. Payor 26 will also have dashboard access to distributed ledger 14 for curated data relevant to claims in facilities with which they are contracted. In embodiments, system 10 allows for the potential direct reimbursement of vendors for their products and implants from a payor, as opposed to reimbursing hospitals for specific expenses related to a claim.

Hospitals and insurance companies (payors) typically work in contract agreements. These agreements allow the hospital or healthcare facility to bill the payor at an agreed upon price for these products used in the facility. It is not untypical to see reimbursements for products, especially implants, at a significant up charge percentage, sometimes 2 to 3 times the cost of what hospitals pay the vendor for the product. There is no transparency on behalf of the payor to what the hospital pays for a product.

In addition, much of the data that is exchanged between healthcare facilities and hospitals is also very difficult to interpret due to coding. A procedure can have numerous codes associated with it, and there are no set standards for billing codes, leaving much to interpretation. No procedure is the same, but certain codes are not indicative of what care was administered. This leads to extremely challenging and time-consuming data mining processes for insurance companies to understand claims and associated reimbursement. This information is crucial in authorization, as payors balance solvency of their combined ratio.

The application creates a place in the ecosystem between hospital or healthcare facility and vendor suppliers. This gives the payor complete visibility of a surgical claim, for example, and the transaction that occurs between the facility and the vendor supplier. The potential to automate claim reimbursement payments direct to vendors exists through system interoperability. In this arrangement, the flow of invoicing will be duplicated directly to the appropriate payor organization for prompt payment. This ensures transparency of these transactions and saves significant costs to the system. All transactions will be stored on a centralized or decentralized ledger.

This arrangement through the application gives the payor complete access to all dashboard data. Examples of the data to be accessible, but not limited to are product or implant, price, total costs of implants by procedure, volume of procedures etc. Pre-authorizations for patient surgeries are delayed and pushed backed even for standard elective cases because of the payor's inability to predict the cost or outcome of a surgery. Even “routine procedures” have variances in the cost outcome. In age, BMI, and other co-morbidities play a role in the outcomes that are unpredictable and cause the payor to delay or deny an authorization of a procedure.

Referring to FIG. 2, beginning from point A, at step 102, a vendor 22 uploads/modifies a product list in system 10. At step 104, the vendor 22 uploads/modifies a price list in system 10. At step 106, a purchase order is received from healthcare facility 12 and at step 108, one or more products are shipped. At step 120, identifying information of the shipped products may be tagged in vendor or device maker's records as “on consignment at step 112, or used at a later time to create a purchase order at step 144.

As the same or a later time, a healthcare facility 12 starting point B downloads Manufacturer Product & Price Lists into Product Data Store 124. The download process may happen in response to a user command, continuously or on a predetermined time schedule.

Independently an employee of healthcare facility 12, beginning from point C, plans a Surgical event at step 130, for example an implant operation, and checks product inventory at step 132. At decision point 134, product data store 124 is updated and a message is created that the product is available and assigned for Surgery at step 136. At step 126, a shipped product is received and scanned.

A surgical event starts at point D and begins with step 140 of a product quality control process. This process may include a number of steps, such as scanning a barcode associated with the product to be used in the surgery at step 142. At step 138, healthcare facility 12 creates a post-surgery product form, which is sent to vender or device maker 22 at step 110. At step 114, the product is tagged as “used” and at step 116, an invoice is created.

At step 128, an automated Re-order Process is initialized which triggers the creation of a Purchase Order at step 144. At step 146, the invoice is approved by healthcare facility 12, at step 148 payment is sent, at step 118, payment is received by vendor 22 and the process ends at point E.

In embodiments, various steps described above are performed using distributed ledger 14. Various modifications of the steps of FIG. 2 with regard to timing and sequence are contemplated.

In embodiments, distributed ledger 14 allows both healthcare facility 12 and vendor 22 to share the same inventory data record. This data record will encompass all data points that are relevant to a product or implant's description. This includes but is not limited to description, company (vendor), reference number, lot number, expiration, born on date, etc. This information is crucial to the PO process and return shipping. Inventory is accounted for by manual scan or upload when being added to the application portal. Facilities and vendors have the ability to add or delete inventory items at their will as described at step 122 of FIG. 2. This process is especially important to replenishment process of inventory used in a. procedure according to step 128.

Vendors 22 typically consign appropriate amounts of product inventory at facilities to decrease operational servicing costs. These consignment agreements allow for inventory stock to live at a facility for as long as a vendor 22 and healthcare facility 12 are partners in business. They can be changed at either party's decision, and are at no cost to arrange. In return, vendor 22 asks that the facility maintain the liability of the product while it is on location. In most situations, there is no oversight or record of what is on location at healthcare facility 12. Most facilities will require a list from the vendor of what inventory is going in the facility on consignment, but it is rarely if ever maintained in accordance with standard procedures. The vendor typically monitors these inventories on their own systems, but with very little visibility to the accuracy in real time as the products are used. The only check currently in place for vendors is routine cycle counts. These audits, if well maintained, are done in time specific increments, but tend to have numerous discrepancies that result in inventory loss and waste accrued to the vendor. Vendor 22 may attempt to reconcile these losses with healthcare facility 12. In situations where healthcare facility 12 maintains inventory it is rarely done in collaboration with vendor 22, and vendor 22 still has the unilateral ability to take this inventory stock at their own will.

This makes the process difficult to manage, and void of any shared transparency and accountability. These all account to more waste and inefficiency in the healthcare system. In many consignment inventory situations, vendors 22 place or purchase space on a shelf or shelves in healthcare facility 12. Vendor Reps can be put in positions where they can add or charge for inventory that was not used in a surgery simply based on what they identify as missing from an instrument set or a par level of implants based on visual inspection. There is no way to truly verify what they say needs to be replaced until it is too late. This leads to many cases of fraud and adds another layer of distrust between vendor 22 and healthcare facility 12.

Distributed ledger 14 creates a baseline of inventory data to be utilized by both healthcare facility 12 and vendor 22 by doing an onsite cycle count or data upload from the facility. All items are tagged as consignment or owned products for differentiation. System 10 also allows for visibility into any inventory that is sent to the facility on a consignment basis. All participating parties as shown in FIG. 1 have the ability to add or remove inventory through distributed ledger 14. Adding or removing inventory requires validation by the other party. System 10 will further expand on the process through the surgical form entry process; when items are used in a surgical procedure they are logged and matched through the application with existing inventory data (steps 142 and 138). These logs may be validated by the vendor representatives designated to handle the information.

Once validated by vendor 22, replenishment stock inventory may be scanned or manually entered and processed (step 108). These items then may be validated by the facility receiving them to ensure accuracy (step 126). The facility will have a list on access and visibility to any “pending inventory” until the physical inventory arrives for validation. The option to “double check” added products can be achieved through an inventory scan by the facility when received. If inventory is scanned during this process that was not on the vendor list then vendor 22 will be alerted. All inventory added in replenishment is redirected back to inventory value of which it originated (consignment, owned or loaned). All incoming inventory orders have the ability to be marked and traced with the purchase order assigned (steps 112 and 114). This can have value for facility tracking replenishment inventory from a particular surgical procedure. Distributed ledger 14 will record only what was used in surgery and notifies of any product or implant wasted or implanted. This gives complete transparency to both parties with real time data visibility and access. When usage is entered the Vendor, and the local hospital network can all see what was used and what needs to be reordered leaving nothing to chance or to subjectivity.

Because all inventory is scanned into system 10, both vendor 22 and healthcare facility 12 have visibility into inventory. In embodiments, an actual contract is automatically generated from the point of use to the point of PO and invoice, vendor 22 can now only replace what the hospital says it used eliminating the discrepancy that may exist. In embodiments, transactions are stored in a centralized or decentralized ledger 14 for transparency.

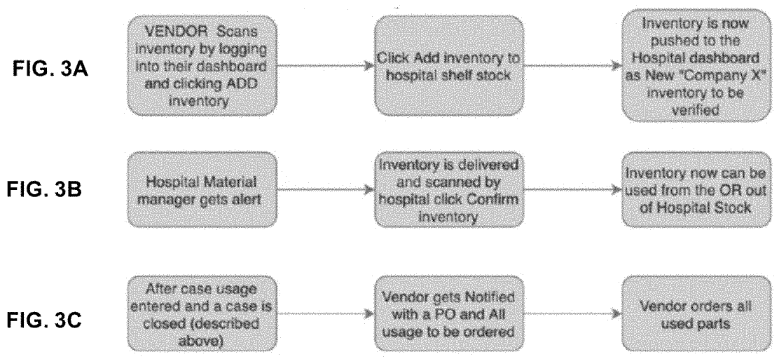

As shown in FIG. 3A, when a user at vendor 22 wants to move product to a healthcare facility 12 consignment inventory, the user may go to the vendor dashboard, select a healthcare facility 12, then click on the button/link for “add inventory.” A screen appears and the user may either scan each inventory piece or upload a file. In embodiments, the user may convert an EXCEL spreadsheet with all of the Lot codes, convert it into JSON and upload it to the system.

As shown in FIG. 3B, once loaded, system 10 may send the healthcare facility an alert letting them know that inventory is being moved together with the list of all of the parts. When healthcare facility 12 receives the product, they can choose to scan it into their inventory or allow the vendor to manage it on their own. If they do not want to scan it they can simply click “confirm inventory”; once they select this entry a contract is issued using a smart contract in distributed ledger 14 that is binding. If healthcare facility 12 chooses to scan the products, they can click “scan in consignment” whereby a screen populates with each piece the facility was notified about. When an implant is scanned it is removed from the list. If something is scanned. that is not on the list the user will receive an alert that says “not a part of this contract.” If the product is scanned, the vendor would be able to verify that what was scanned by the healthcare facility matched what they scanned into their inventory. If everything matches the facility can click “confirm inventory”. Once all of the inventory is confirmed the vendor and the facility would have real time visibility to their inventory.

As shown in FIG. 3C, when a product is used, it is scanned at healthcare facility 12, vendor 22 is notified and the product is replaced automatically.

Using Smart contracts 18 of distributed ledger 14, system 10 may identify products that are unsafe or not intended for use on a particular patient during surgery. Utilizing implant inventory data specific to each account, system 10 will ensure conditions are met for each product intended for implantation or use. In embodiments, a surgery is scheduled using a product or implant designated “anatomic right”. When scanned, system 10 will detect whether or not the implant or product is an “anatomic right” in accordance with the surgical site (right, left, axial etc.). If the implant upon scanning, the implant was determined to be designated “anatomic left”, system 10 would alert the user that this is an incorrect product or implant choice. These same conditional requirements may also be applied for expiration, Instructions For Use (IFUs) or prescribed use. In the event of prescribed use, a preloaded set of requirements can be input prior to a procedure. If a product or implant is scanned or manually entered prior to being used and did not meet the prescribed requirements, it would alert the surgical team.

In an operating room during surgery, when it is time to open an implant, a Nurse or Vendor rep is told by the surgeon what implant to select from the available stock to use in the patient on the table. Selection of an implant or product often relies only on verbal instructions and visual confirmation which may be prone to mistakes. Typically, there are an average of 40 wrong site or wrong product procedures done every week in the US. This is a major patient safety issue that is still reliant on human eye and verbal authentication. In conventional systems, situations exist where these items are opened for use incorrectly, but not implanted. Estimates are in the thousands for implants or products that are consumed due to human error every month. This leads to massive amounts of healthcare waste on behalf of the vendor and the facility.

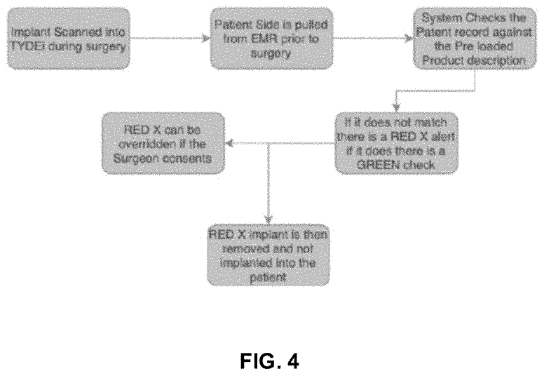

FIG. 4 illustrates a representative product safety process as part of a healthcare inventory management distributed ledger system, in embodiments. A surgical procedure on a patient's right knee is set to be performed. An implant is selected intra-operatively and before it is opened it is scanned into the application system. The system verifies the implant inventory origination with the supply chain (consignment, loaned by vendor for use or owned by the facility). If the item is expired or nonconforming based on surgical site, conditional requirements will not be met, and an alert will be made to a user. If the implant or product is indeed for the right knee and all conditional requirements have been met the user will be alerted. This is done to ensure safety and efficacy of the implant or product selected to be used on or in a patient. If the requirements are not met during a surgical procedure based on alert, it is the user's responsibility to ensure that an implant or product that meets requirements is used. The incorrect item can be struck from the record in the system but will not be charged or wasted because it was not used. In the case of a surgery using a prescribed product or implant(s), it is the responsibility of the physician to ensure that the correct product or implant has been pre-authorized and prescribed by the system. When ready inter-operatively, user would scan or enter product or implant information into the system for authentication. If it is not a match there would be an alert by the application for notification. If that same process meets all requirements that have been pre-authorized in accordance with the provider's specifications, the user will be alerted that the product or implant is safe to use in the procedure.

In embodiments, system 10 will perform validation between the characteristics of the Surgical Event and the characteristics of the intended product for implantation. These validations include but are not limited to the Expiration date comparison and the Anatomic side comparison between the product and the surgical event procedure. The system will warn of any discrepancy and record the event, decision (accept or Decline), user of the system, and date on the encrypted distributed ledger 14 using smart contracts 18. The system will maintain these trusted and immutable records which can be viewed by any authorized users to the system. Due to its immutable characteristics of the distributed ledger 14, no record can be deleted or tampered with. All records and messages will be accessible through various means including computer, mobile devices and/or electronic readers.

Utilizing smart contracts, system 10 may automatically issue and generate a PO from point of entry to the specific vendor or multiple vendors simultaneously. The application also allows for an option to have the smart contract verified by any other user that would be required to authorize or authenticate the PO due to requirements. These contracts can be tailored conditionally specific to the facility or hospital.

In the operating room there is typically an implant sheet that is given to vendors for the purposes of reordering implants and verifying payment for PO purposes. Because there is no system that allows vendors to connect to the system of healthcare facility 12, they are forced to take patient information (name, DOB, MR #, Procedure side, procedure type, implants put in, with pricing) and email it within their system of records with the “promise” that they will protect the privacy of the information. In addition to sending patient information in an email, vendor 22 may also use the patients name or MR number in order to file it in their implant ordering systems as well. Further, the same sheet of paper pass through the hands of up to 6 different people at healthcare facility 12 who each make separate copies of it. In embodiments, system 10 eliminates that set of redundant processes as well as supports HIPAA compliance.

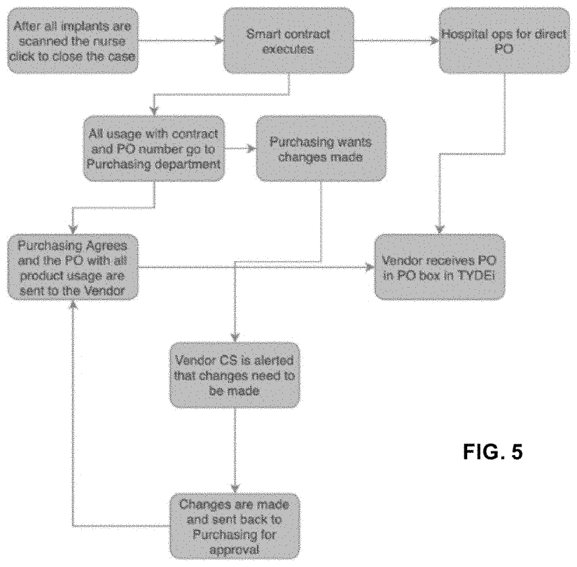

FIG. 5 illustrates a representative purchase order as part of a healthcare inventory management distributed ledger system, in embodiments. During a surgical procedure, system 10 can pull information direct from electronic medical record (EMR) or electronic health record (HER) data to ensure correct surgery, patient and surgical site side. If not connected to EMR or EHR, information may be entered into system 10 manually. Used products are scanned data or manually input necessary product information (Ref #, Lott #, expiration date, description, etc.). Using smart contracts, system 10 will verify validity of product being used to ensure safety through conditional requirements. Some of these requirements are, but are not limited to anatomic implants, expiration, prescribed implants, on-label use in accordance with vendor's IFU, etc.

When the log for surgery is completed and all items used in a procedure have been accounted for the user will validate and authorize the form. System 10 will only process the form if conditional requirements have been fulfilled. Assuming all requirements have been met, the system will then automate a purchase order to the specific vendors who supplied the products. The purchase order may be received and verified by vendor representative to continue remittance process. The form will cryptographically encrypt all sensitive information (PII, PHI etc.) with a hash code for identification purposes to ensure that the vendor does not receive sensitive identifiable information.

Each healthcare facility 12 will grant access to users tasked with managing input into the system. Each user will be provided with a secure log-on into system 10 for access that can be controlled by operator. All vendor contracts are individually loaded into the system. Each vendor that a facility does business with will have access granted to them by the hospital utilizing the application. This includes, but is not limited to customer service, accounts receivable, any administrative staff, purchasing agents, material managers, operations, etc. This method of controlling users both by the hospital and vendors allows for a closed communication portal for all transactions. Those groups with access are the only groups permitted to operate within the ecosystem.

In embodiments, system 10 may determine if an implant or product that has been used has been recalled by supplier or vendor. If an implant or product recall is issued vendor 22 is required to notify healthcare facilities 12 of all effected lots purchased by the healthcare facility. System 10 will identify all affected product or reference numbers and lot numbers can be accounted for through search in the data vault of a particular facility. A simple search of this information will locate all specific patients impacted. A list of all patients with a problematic lot can be curated for risk management to notify surgeons and patients. These patients may require additional observation or treatment. This allows for a quick and simple response to these issues, and allows contact to be initiated with all patients. This can be achieved by logging an electronic format of effected reference and lot numbers or manually entering data sourced from vendor.

When a manufacturer issues a recall of products that have been implanted into patients, they issue warning letters to each implanting physician and each facility where they were implanted. This is typically done by paper record and attached to the warning is a list of every affected Lot number that needs to be addressed. There are no patient names, implantation dates or any real identifying information that helps the hospital identify the patients that may need to be contacted. The Hospital would just work with the surgeon toward their best guess and a manual implant look up in the EMR for every patient they think may be affected. The most information a vendor provides is an invoice date not a surgery date. This can narrow down the date of surgery too within a couple months but still makes for a terribly inefficient process.

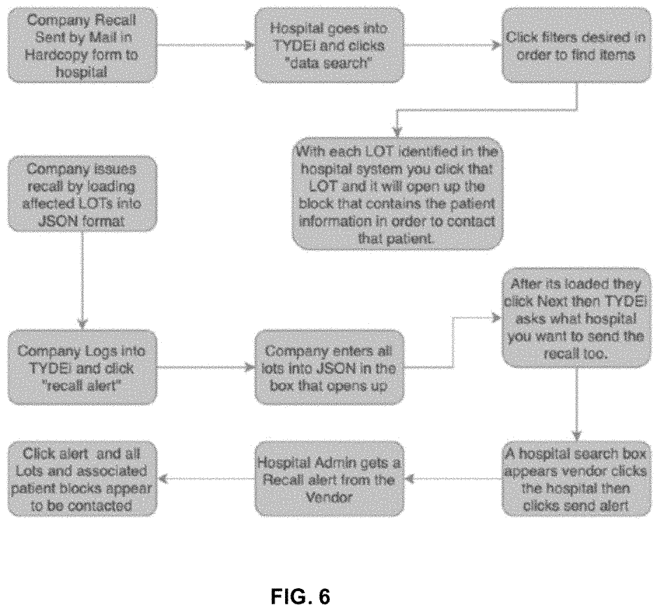

FIG. 6 illustrates a representative recall process as part of a healthcare inventory management distributed ledger system, in embodiments. Company X recalls a Hip implant that was implanted in 50,000 total patients. These hip implants need to be removed or they could cause patient harm. Vendor 22 using system 10 has a number of options. In embodiments, vendor 22 may upload the data file into the application and alert each hospital as to what patient lots are affected. Each affected hospital has a different list of affected patients and they can simply upload that recall file in JSON and connect it to the desired hospital. When that hospital Administrator is pinged with the recall data they can automatically pull up each patient's record based on the reference number and lot code provided by the vendor. Now the hospital can reach out to each patient and to the surgeon responsible for care. In embodiments, vendor 22 may send a curated list of Reference numbers and Lots and healthcare facility 12 may retrospectively research each individual item included in the recall attached to a patient log.

### FHIR, HL7, EMR, HER and ERP Integration and Interoperability

In embodiments, system 10 including centralized or decentralized distributed ledger 14 has full interoperability with existing software services, programs etc. In order to achieve a full end to end experience for facilities and partnered vendors, system 10 can both read and write into all existing systems within a hospital. Some examples would include, but are not limited to: EHR, EMR, FHIR servers, ERP, etc. System 10 acts as an overlay system that will interact with all existing systems in a facility.

System 10 will pull data from EMR, EHR and FHIR servers, ERP etc. to retrieve relevant PHI or PII to a surgical procedure. After a product/implant log is completed in the application, the data can be shared through any EMR, EHR, FHIR server, ERP etc.

In another embodiment, a dashboard will allow anyone given access to see all chosen data, but the way it is organized is key for a researcher or insurance companies.

Often billing notations as seen by an Insurance company will be cryptic, such as a procedure described as “on July 4^{th}—1942 CPT code 456 was charged or billed.” This causes discrepancies in understanding what was billed why and for how much. It also does not allow for an understanding around waste that may have been generated in one case versus another. In other situations, instead of using the CPT code format, a procedure may be described with a text string such as “Total Knee was performed.”

Using healthcare inventory management system 10, a user would be able to see much more data, both at an individual patient level and usage of products across healthcare facilities, surgeons, companies, geographical areas or by other grouping criteria. A user may also observer whether or not there were wasted implants and disposable charges. Mathematical formulas could be applied such as the cost average per surgery. All information that can be used for insurance.

System 10 provides for advanced communication between user, for example, a “chat” format of communication between a hospital and a vendor (or between any 2 entities), where it is necessary to protect sensitive customer or patient information (PII, PHI). All identifiable information is encrypted during communication displaying only a “hash” identifier in its place. All transcripts of communication are logged in encrypted, distributed ledger 14 (either centralized or decentralized) for future reference.

Current communication methods are limited to paper ledger, fax, email, text etc. with information displayed to end users who may not have authorization to view or access that information. Some entities do attempt to use encrypted communication (such as e-mail), but identifiable information is still made available. There are no measures of accountability to determine if vendor personnel are HIPAA trained, improperly handle sensitive information in respect to HIPAA compliance or have had background checks to ensure they are not bad actors. The large volume of communication and messages that occur between a facility and vendor representative often involve bad practices susceptible to fraudulent activity by any party, including representatives of a hospital, vendor, or payor.

Various entities may transmit messages through the application's chat functionality via text, PDF, JPEG or any other visual format in order to avoid any current standards of communication (email, text or phone calls). This ensures security around sensitive identifiable information and creates a recorded log for shared accountability between any representatives with access. The lack of a direct encrypted communication network between vendors and hospitals results in an unbelievable amount of communication that does not protect sensitive information and allows visibility to those who should not be granted access. The potential for vendor mishandling of this sensitive information creates a significant liability.

System 10 may provide messenger functions in an intuitive way. A menu may allow selection by individuals or groups according to categories such as vendor, vendor division or other healthcare facility networks. Then click on 1 or all of them to send them a message in a thread where they can all see the same data simultaneously. You can even create files to organize your conversations. The system is immutable and transparent to all parties as the smart contract determines That means that the operator is able to determine who is allowed to see a thread based on the facilities preferences they determined in their smart contract.

In embodiments, a medical professional such as a doctor may order implants are needed for surgery directly using a Smart Contract 18. For example, in the field of Ophthalmology, a surgeon could enter a prescribed lens for implanting into a patient into system 10. System 10 would then create a compliance rule via smart contract technology that would be able to verify in the operating room that the lens brought to the operating room is the correct lens that was ordered from the physician's office.

In a further embodiment, if a surgeon puts the patients needed prescriptions in system 10 upon entry and completion the system would then create a specific contract for that patient. Before surgery the hospital or surgery center would be able to see if the correctly ordered implant is available in the facility (this would also be visible to the vendor). After that is verified as available the nurse in the facility would pick the ordered implant. When the surgeon directs the nurse to open the implant the nurse would first scan the item. If the item is not correct according to what the surgeon ordered. A compliance warning would populate on the screen warning the OR team not to use that implant. If it is correct everything would carry on as normal

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A computer-implemented method for managing product inventory using a distributed ledger, the method comprising:
receiving inventory data comprising a product list from a vendor;
storing the inventory data as records in the distributed ledger;
executing a first smart contract in the distributed ledger to process a request to use a selected product from the product list;
executing a second smart contract in the distributed ledger to record the completion of use of the selected product; and
executing a third smart contract to complete a payment process to the vendor.

2. The method of claim 1, wherein executing the first smart contract further comprises checking the inventory data records in the distributed ledger to see if the selected product is available for use.

3. The method of claim 1, wherein executing the second smart contract further comprises scanning a barcode on the selected product before it is used in a surgical procedure.

4. The method of claim 3, wherein executing the second smart contract further comprises verifying that the selected product is appropriate for the surgical procedure.

5. The method of claim 3, wherein executing the second smart contract further comprises:
scanning the barcode on the selected product after the surgical procedure; and
storing a record in the distributed ledger.

6. The method of claim 1, wherein executing the third smart contract further comprises:
generating a purchase order;
creating an invoice based on the purchase order; and
updating the inventory data records in the distributed ledger.

7. The method of claim 1, further comprising encrypting all records before they are stored in the distributed ledger.

8. A system for healthcare inventory management for managing product inventory using a. distributed ledger, the system comprising a processor and a data store storing data and instructions which, when executed, cause the processor to:
receive inventory data comprising a product list from a vendor;
store the inventory data as records in the distributed ledger;
execute a first smart contract in the distributed ledger to process a request to use a selected product from the product list;
execute a second smart contract in the distributed ledger to record the completion of use of the selected product; and
execute a third smart contract to complete a payment process to the vendor.

9. The system of claim 8, wherein executing the first smart contract further comprises checking the inventory data records in the distributed ledger to see if the selected product is available for use.

10. The system of claim 8, wherein executing the second smart contract further comprises scanning a barcode on the selected product before it is used in a surgical procedure.

11. The system of claim 10, wherein executing the second smart contract further comprises verifying that the selected product is appropriate for the surgical procedure.

12. The system of claim 10, wherein executing the second smart contract further comprises:
scanning the barcode on the selected product after the surgical procedure; and
storing a record in the distributed ledger.

13. The system of claim 8, wherein executing the third smart contract further comprises:
generating a purchase order;
creating an invoice based on the purchase order; and
updating the inventory data records in the distributed ledger.

14. The system of claim 8, further comprising encrypting all records before they are stored in the distributed ledger.

15. A non-transitory computer-readable medium, having stored thereon program instructions that, upon execution by a processor, cause the processor to perform a method comprising:
store the inventory data as records in the distributed ledger;
execute a first smart contract in the distributed ledger to process a request to use a selected product from the product list;
execute a second smart contract in the distributed ledger to record the completion of use of the selected product; and
execute a third smart contract to complete a payment process to the vendor.
